(19) United States
(12) Patent Application Publication
(10) Pub. No.: US 2004/0002757 A1
(43) Pub. Date: Jan. 1, 2004

(54) Intraocular lens

(75) Inventors: Yu-Chin Lai, Pittsford, NY (US); Dominic V. Ruscio, Webster, NY (US); David P. Vanderbilt, Webster, NY (US)

Correspondence Address: Bausch & Lomb Incorporated, One Bausch & Lomb Place, Rochester, NY, 14604-2701, US

(73) Assignee: Bausch & Lomb Incorporated, Rochester, NY

(21) Appl. No.: 10/185,520

(22) Filed: Jun. 27, 2002

(51) Int. Cl. A61F 2/16

(52) U.S. Cl. 623/6.16

(57) ABSTRACT

An intraocular lens for inhibiting posterior capsular opacification, or secondary cataract, includes an optic having a periphery provided with a sharp, flexible flange which presses against the posterior capsule wall thereby creating a barrier to LEC migration.

### Background Of The Invention

The present invention relates to intraocular lenses (IOLs) for implantation in an aphakic eye where the natural lens has been removed due to damage or disease (e.g., a cataractous lens). The present invention more particularly relates to a novel IOL designed to inhibit the unwanted growth of lens epithelial cells (LECs) between the IOL and posterior capsular bag, also known as posterior capsule opacification or “PCO” to those skilled in the art.

A common and desirable method of treating a cataract eye is to remove the clouded, natural lens and replace it with an artificial IOL in a surgical procedure known as cataract extraction. In the extracapsular extraction method, the natural lens is removed from the capsular bag while leaving the posterior part of the capsular bag (and preferably at least part of the anterior part of the capsular bag) in place within the eye. In this instance, the capsular bag remains anchored to the eye's ciliary body through the zonular fibers. In an alternate procedure known as intracapsular extraction, both the lens and capsular bag are removed in their entirety by severing the zonular fibers and replaced with an IOL which must be anchored within the eye absent the capsular bag. The intracapsular extraction method is considered less attractive as compared to the extracapsular extraction method since in the extracapsular method, the capsular bag remains attached to the eye's ciliary body and thus provides a natural centering and locating means for the IOL within the eye. The capsular bag also continues its function of providing a natural barrier between the aqueous humor at the front of the eye and the vitreous humor at the rear of the eye.

One known problem with extracapsular cataract extraction is posterior capsule opacification, or secondary cataract, where proliferation and migration of lens epithelial cells occur along the posterior capsule behind the IOL posterior surface which creates an opacification of the capsule along the optical axis. This requires subsequent surgery, such as an Nd:YAG laser capsulotomy, to open the posterior capsule and thereby clear the optical axis. Undesirable complications may follow the capsulotomy. For example, since the posterior capsule provides a natural barrier between the back of the eye vitreous humor and front of the eye aqueous humor, removal of the posterior capsule allows the vitreous humor to migrate into the aqueous humor which can result in serious, sight-threatening complications. It is therefore highly desirable to prevent posterior capsule opacification in the first place and thereby obviate the need for a subsequent posterior capsulotomy.

Various methods have been proposed in the art to prevent or at least minimize PCO and thus also the number of Nd:YAG laser capsultomies required as a result of PCO. These PCO prevention methods include two main categories: mechanical means and pharmaceutical means.

In the mechanical means category of PCO prevention, efforts have been directed at creating a sharp, discontinuous bend in the posterior capsule wall which is widely recognized by those skilled in the art as an effective method for minimizing PCO. See, for example, Posterior Capsule Opacification by Nishi, Journal of Cataract & Refractive Surgery, Vol. 25, January 1999. This discontinuous bend in the posterior capsule wall can be created using an IOL having a posterior edge which forms a sharp edge with the peripheral wall of the IOL.

In the pharmaceutical means of PCO prevention, it has been proposed to eliminate LEC and/or inhibit LEC mitosis by using an LEC-targeted pharmaceutical agent. See, for example, U.S. Pat. No. 5,620,013 to Bretton entitled “Method For Destroying Residual Lens Epithelial Cells”. While this approach is logical in theory, putting such a method into clinical practice is difficult due to complications arising, for example, from the toxicity of some of the LEC inhibiting agents themselves (e.g., saporin), as well as the difficulty in ensuring a total kill of all LECs in the capsular bag. Any remaining LECs may eventually multiply and migrate over the IOL, eventually resulting in PCO despite the attempt at LEC removal at the time of surgery.

By far the most promising method for inhibiting LEC formation on the posterior surface of an IOL is the mechanical means, i.e., by designing the IOL to have a sharp peripheral edge particularly at the posterior surface—peripheral edge juncture to create a discontinuous bend in the posterior capsule wall. This discontinuous bend in the posterior capsule wall has been clinically proven to inhibit the growth and migration of LECs past this bend and along the IOL surface. One of the early reports of this PCO-inhibiting effect of a planoconvex IOL may be found in Explanation of Endocapsule Posterior Chamber Lens After Spontaneous Posterior Dislocation by Nishi et al, J Cataract & Refractive Surgery—Vol 22, March 1996 at page 273 wherein the authors examined an explanated planoconvex PMMA IOL where the posterior surface of the IOL was planar and formed a square edge with the peripheral edge of the IOL:

“Macroscopic view of the explanted IOL and capsule revealed a 9.5 mm capsule diameter. The open circular loops fit well along the capsule equator. The capsule equator not in contact with the haptic was also well maintained (FIG. 3). An opaque lens mass (Soemmering's ring cataract) was seen between the haptics and optic. The posterior capsule facing the IOL optic was clear.

Histopathological examination of the explanted capsule revealed few epithelial cells (LECs) on the posterior capsule. Between the loops and the optic, a lens mass with accumulation at the edge of the optic was seen (FIG. 4). There was an obvious bend in the posterior capsule at this site.” (Emphasis added.)

Thus, in the years since this report, the industry has seen much activity on creating IOLs with sharp posterior edges so as to create a sharp, discontinuous bend in the posterior capsule wall. While IOLs having a sharp posterior edge have proven to inhibit PCO compared to IOLs having rounded edges at the posterior surface-peripheral edge juncture, there still remains the possibility of LECs migrating along the posterior capsule and behind the IOL surface, especially if there is uneven contact and force of the IOL periphery with the capsular bag. This may happen, for example, should the IOL move within the capsular bag following surgery. There therefore remains a need for an improved IOL design which addresses the problem of LEC migration and PCO formation.

### SUMMARY OF THE INVENTION

The present invention addresses the problem of PCO by providing an IOL having an optic with an optic peripheral flange which is configured much thinner and more flexible than respective peripheral edges of IOLs of the prior art. In particular, prior art IOLs typically have an edge thickness of about 240 microns and are relatively rigid. Conversely, the present inventive IOL has an optic formed with a thin, flexible flange which is preferably about 25 to about 120 microns thick and about 25 to 120 in width. The thin, flexible flange extends around the entire periphery of the optic and engages the posterior capsule when the IOL is implanted in the eye. The thin, flexible flange of the IOL acts as a barrier to migration of lens epithelial cells across the posterior capsule and therefore acts to inhibit PCO. This configuration of the periphery of the IOL optic is a significant improvement over the prior art IOLs having PCO inhibition features in that it provides an improved barrier against LEC migration. The following are patents and publications which show various IOL optic periphery designs:

U.S. Pat. No. 5,171,320 issued to Nishi on Dec. 15, 1992

U.S. Pat. No. 5,693,093 issued to Woffinden F T al on Dec. 2, 1997

U.S. Pat. No. 6,162,249 issued to Deacon F T al on Dec. 19, 2000

### BRIEF DESCRIPTION OF THE DRAWING

FIG. 1 is a cross-sectional view of a human eye showing the natural lens within the capsular bag of the eye;

FIG. 2 is a cross-sectional view of a human eye showing the natural lens removed and replaced with a prior art IOL;

FIG. 3 is a plan view of a prior art IOL;

FIG. 4 is a plan view of an IOL made in accordance with the present invention;

FIG. 5 is an enlarged, fragmented, cross-sectional view showing the detail of the peripheral flange configuration of the IOL of the present invention engaged with the posterior capsule in the intended manner; and

FIG. 6 is a simplified cross-sectional viewof the IOL as taken along the line 6-6 of FIG. 4 and showing placement of the IOL of the present invention in the capsular bag of the eye.

### DETAILED DESCRIPTION

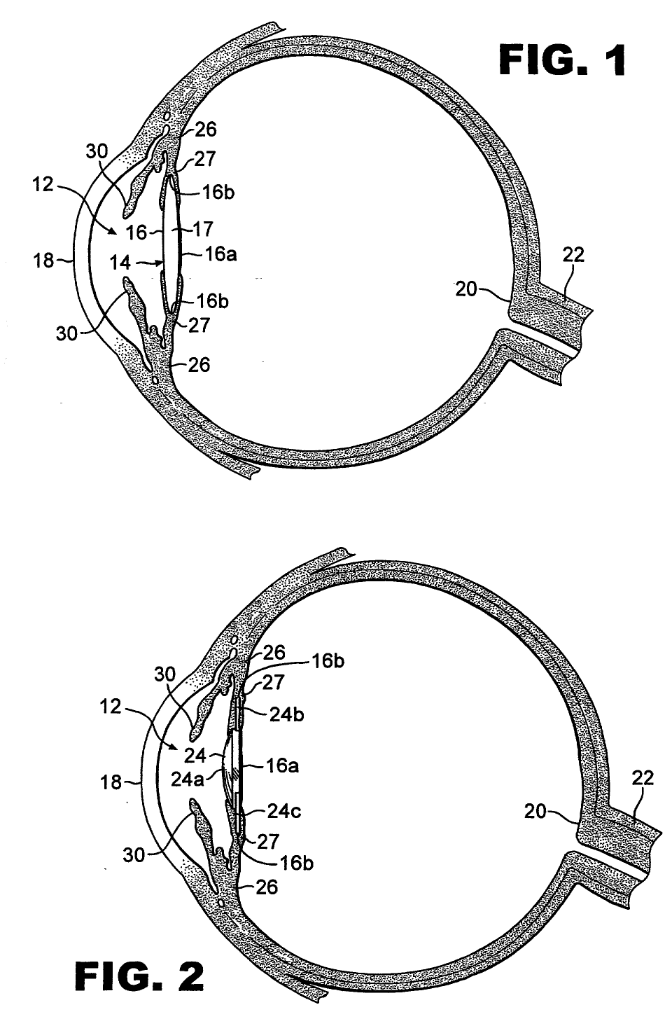

Referring now to the drawing, there is seen in FIG. 1 a cross-sectional view of a human eye 10 having an anterior chamber 12 and a posterior chamber 14 separated by the iris 30. Within the posterior chamber 14 is a capsule 16 which holds the eye's natural crystalline lens 17. Light enters the eye by passing through the cornea 18 to the crystalline lens 17 which act together to direct and focus the light upon the retina 20 located at the back of the eye. The retina connects to the optic nerve 22 which transmits the image received by the retina to the brain for interpretation of the image.

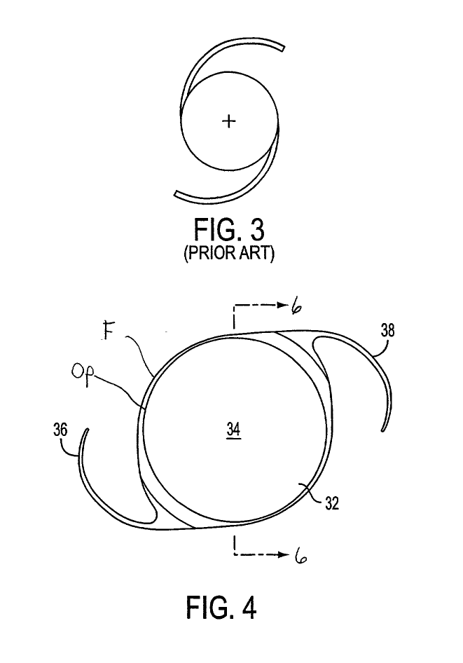

In an eye where the natural crystalline lens has been damaged (e.g., clouded by cataracts), the natural lens is no longer able to properly focus and direct incoming light to the retina and images become blurred. A well known surgical technique to remedy this situation involves removal of the damaged crystalline lens which may be replaced with an artificial lens known as an intraocular lens or IOL such as prior art IOL 24 seen in FIGS. 2 and 3. Although there are many different IOL designs as well as many different options as to exact placement of an IOL within an eye, the present invention concerns itself with an IOL for implanting inside the substantially ovoid-shaped capsule 16 of eye 10. This implantation technique is commonly referred to in the art as the “in-the-bag” technique. In this surgical technique, a part of the anterior portion of the capsular bag is cut away (termed a “capsularhexis”) while leaving the posterior capsule 16a intact and still secured to the ciliary body 26.

Thus, in the “in-the-bag” technique of IOL surgery, the IOL is placed inside the capsule 16 which is located behind the iris 30 in the posterior chamber 14 of the eye. An IOL includes a central optic portion 24a which simulates the extracted natural lens by directing and focusing light upon the retina, and further includes a means for securing the optic in proper position within the capsular bag. A common IOL structure for securing the optic is called a haptic which is a resilient structure extending radially outwardly from the periphery of the optic. In a particularly common IOL design, two haptics 24b, 24c extend from opposite sides of the optic and curve to provide a biasing force against the inside of the capsule which secures the optic in the proper position within the capsule (see FIG. 2).

As stated in the Background section hereof, an undesirable post-surgical condition known as posterior capsule opacification or PCO may occur which results in an implanted IOL becoming clouded and thus no longer able to properly direct and focus light therethrough. The main cause for this condition is the mitosis and migration of lens epithelial cells (LECs) across the posterior surface of the capsule behind the IOL optic. As seen in FIG. 2, the posterior surface 16a of the capsule 16 touches the posterior surface of the IOL optic 24a. When the damaged natural lens is surgically removed, a number of LECs may remain within the capsule 16, particularly at the equator 16b thereof which is the principle source of germinal LECs. Although a surgeon may attempt to remove all LECs from the capsular bag at the time of IOL implantation surgery, it is nearly impossible to remove every single LEC. Any remaining LECs can multiply and migrate along the posterior capsule wall 16a. This is especially true in IOLs having rounded flanges, where it has been found that clinically significant PCO results in about 20%-50% of patients three years post surgery. A presently popular and effective method of preventing PCO is to create a sharp, discontinuous bend in the posterior capsule wall 16a as explained in the Background section hereof.

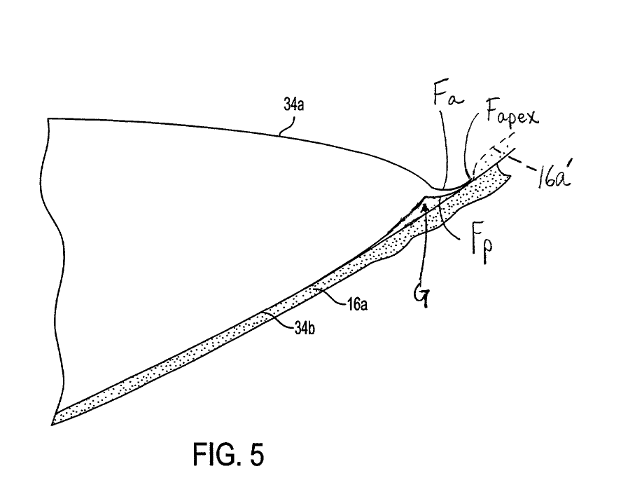

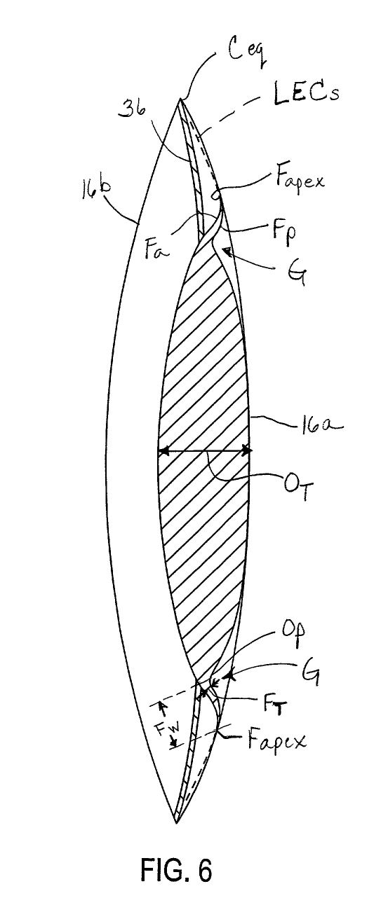

Referring now to FIGS. 4 and 5, a first embodiment of the inventive IOL 32 is shown. IOL 32 is seen to include a central optic portion 34 having opposite anterior and posterior surfaces 34a and 34b, respectively. When implanted within the eye, anterior optic surface 34a faces the cornea 18 and posterior optic surface 34b faces the retina 20. A pair of haptics 36,38 are attached to and extend from opposite sides of the periphery of optic portion 34 and are configured to provide a biasing force against the interior of the capsule 16 to properly position IOL 32 therein. More particularly, the haptics 36,38 are configured such that upon implanting the IOL with the capsular bag, the haptics engage the interior surface of the capsular bag as seen in FIG. 2. The engagement between the haptics and capsule creates a biasing force causing the IOL optic 34 to vault posteriorly toward the retina 20 whereupon the posterior surface 34b of the IOL optic presses tightly against the interior of the posterior capsule wall 16a of capsule 16. It is noted that other known IOL positioning means are possible and within the scope of the invention. Furthermore, IOL 32 may be made from any suitable IOL material, e.g., PMMA, silicone, hydrogels and composites thereof The IOL 32 may also be a one piece or multiple piece design (e.g. where the haptics are separately formed and attached to the optic.)

Referring still to FIGS. 4 and 5, it is seen that IOL optic 34 includes an optic periphery O_{P} and a sharp, flexible peripheral flange F which extends radially outward of the IOL optic 34. With the haptics 36,38 providing the biasing force explained above, the optic posterior surface 34b presses against the posterior capsule wall 16a and the sharp peripheral flange F of the IOL optic also presses against the posterior capsule wall 16a. The flange F is flexible as stated above and is also supple such that it will not injure the capsular wall tissue. In this regard, it is noted that the thickness of flange F_{T} as compared to the respective thickness O_{T} of the optic 34 at its thickest point is very small. As stated above, prior art IOLs typically have an edge thickness of about 240 microns and are relatively rigid. Conversely, the present inventive IOL has an optic formed with a thin, flexible flange F which has a thickness F_{T} in the range of about 25 to about 120 microns thick. As such, the flange F will have a much lower stiffness than the optic.

As seen best in FIGS. 5 and 6, the posterior surface F_{P} of flange F presses against capsule wall 16a with the flange apex Fₐₚₑₓ thereof extending radially outwardly of the optic in the direction of the capsule equator C_{eq}. As mentioned above, the primary source of germinating LECs is at the equator C_{eq} of the capsular bag which is located radially outwardly of the optic periphery (FIG. 6). As LECs multiply, they begin migrating radially inwardly along the capsular bag. With the inventive IOL 32 implanted within the eye, LECs migrating from the capsular equator C_{eq} toward the IOL 32 encounter flange apex Fₐₚₑₓ which acts as a barrier to inhibit LEC migration past this point (i.e., between the posterior capsule wall 16a and IOL posterior surface 34b) and PCO is inhibited. It is noted that the flange F may actually indent into the capsule wall and create a bend in the capsule wall indicated by the dashed lines and reference numeral 16a′ in FIG. 5. In this situation, PCO is still inhibited due to the barrier effect of flange F and the bend in the capsular wall created through the interaction of the flange F with the capsule wall.

As seen in FIGS. 5 and 6, when flange F is pressing against the capsular wall 16a, the anterior surface of the flange Fa curves inwardly toward flange posterior surface Fp while the posterior surface Fp bows outwardly to conform to the shape of the posterior capsule surface 16a. In cross-section, the configuration of flange F when pressing against the capsule wall may be characterized as arcuate shaped with the thickness F_{T} thereof being largest directly adjacent the optic 34 and tapering inwardly to the flange apex Fp. A gap G may form between the IOL and capsular wall in the area between the flange F and the optic posterior surface 34b. This is due primarily to this geometry of the flange F with respect to the geometry of the IOL optic 34 which ensures the flange F will rest firmly against the posterior capsule wall 16a.

A presently preferred method of forming the sharp flange configuration in the IOL optic 34 comprises lathing and/or milling operation where the IOL optic is mounted to a fixture and a lathe and/or mill is used to cut the IOL geometry including flange F. Other methods which may be employed to form the peripheral flange geometry includes molding, for example. It is also preferred that the flange F is protected during polishing of IOL 32 so as to ensure the flange F retains its original geometry.

It is also noted that the IOL may be made of any suitable material including, but not limited to, hydrogels, silicones, PMMA, and combinations thereof For example, IOL 34 may have an optic formed of one material, a flange F made of another material and haptics made of the same material as the optic or flange F, or a different, third material.

This unique peripheral flange configuration provides an IOL which substantially inhibits PCO as described above.

What is claimed is:

1. An intraocular lens for implanting in a human eye, comprising:
a) a lens optic having opposite anterior and posterior surfaces defined by an optic periphery; and
b) a flexible flange formed adjacent to and radially outwardly of said optic periphery, said flange having a posterior surface which engages the posterior capsule wall when said IOL is implanted in the eye, said flange further including a flange apex which extends radially outward of said optic and acts as a barrier to prevent lens epithelial cell migration between said posterior surface of said optic and the posterior capsule wall of the eye.

2. The intraocular lens of claim 1, and further comprising means for positioning said intraocular lens within a human eye.

3. The intraocular lens of claim 2 wherein said positioning means comprises one or more haptics extending from said optic periphery.

4. The intraocular lens of claim 3, wherein said haptics apply a biasing force against said optic in the direction of said posterior optic surface upon implanting said intraocular lens in said human eye.

5. The intraocular lens of claim 1, wherein said flange has an flange thickness in the range of about 25 to about 120 microns.

6. The intraocular lens of claim 1, wherein said flange includes an anterior surface and a posterior surface, and wherein said flange posterior surface bows posteriorly such that said flange posterior surface firmly engages the posterior capsule wall of the eye upon implanting said intraocular lens in the eye.

7. The intraocular lens of claim 6, wherein said flange anterior surface curves inwardly whereby said flange is substantially arcuate shaped in cross-section.

8. The intraocular lens of claim 1 wherein said flange tapers inwardly from a point adjacent said optic periphery to said flange apex.

9. An intraocular lens for implanting in a human eye, comprising:
a) a lens optic having opposite anterior and posterior surfaces surrounded by an optic periphery; and
b) a flexible flange formed adjacent to and extending radially outwardly of said optic periphery, said flange having a posterior surface which engages the posterior capsule wall when said IOL is implanted in the eye, said flange further including a flange apex which extends in a direction away from said optic, said flange having an arcuate shaped cross-section, said flange acting as a barrier to prevent lens epithelial cell migration between said posterior surface of said optic and the posterior capsule wall of the eye.

10. The intraocular lens of claim 1 wherein a gap is formed between the capsular wall of the eye and said IOL at the juncture of the flange and the optic periphery.

11. An intraocular lens for implanting in a human eye, comprising:
a) a lens optic having opposite anterior and posterior surfaces surrounded by an optic periphery; and
b) a flexible flange formed adjacent to and extending radially outwardly of said optic periphery, said flange having a posterior surface which engages the posterior capsule wall when said IOL is implanted in the eye, said flange tapering from said optic periphery and terminating in a flange apex which extends in a direction away from said optic, said flange acting as a barrier to prevent lens epithelial cell migration between said posterior surface of said optic and the posterior capsule wall of the eye when said intraocular lens is implanted in the eye.
